(19) United States
(12) Patent Application Publication
(10) Pub. No.: US 2005/0125049 A1
(43) Pub. Date: Jun. 9, 2005

(54) Tissue engineered vascular construct and method for producing same

(75) Inventors: David L. Brown, Ann Arbor, MI (US); Gregory H. Borschel, Ypsilanti, MI (US); Robert G. Dennis, Chapel Hill, NC (US)

Correspondence Address: BROOKS KUSHMAN P.C., 1000 TOWN CENTER, TWENTY-SECOND FLOOR, SOUTHFIELD, MI, 48075, US

(73) Assignee: The Regents of the University of Michigan, Ann Arbor, MI (US)

(21) Appl. No.: 10/970,537

(22) Filed: Oct. 21, 2004

Related U.S. Application Data
(60) Provisional application No. 60/513,004, filed on Oct. 21, 2003.

(51) Int. Cl. A61F 2/06; A61B 17/08

(52) U.S. Cl. 623/1.1

(57) ABSTRACT

A small diameter vascular construct and method for producing the same includes a vessel harvested from a donor and having an internal diameter of less than about 3 mm. The vessel is acellularized to remove cellular elements while retaining an extracellular matrix of the vessel. The vascular construct further includes endothelial cells provided in contact with the acellularized vessel, wherein the endothelial cells attach to the vessel to form the vascular construct.

### CROSS-REFERENCE TO RELATED APPLICATIONS

This application claims the benefit of U.S. provisional application Ser. No. 60/513,004filed Oct. 21, 2003.

### STATEMENT REGARDING FEDERALLY SPONSORED RESEARCH OR DEVELOPMENT

The invention was made with Government support under Contract No. N66001-02-C-8034 from DARPA. The Government has certain rights to the invention.

### BACKGROUND OF THE INVENTION

1. Field of the Invention

This invention relates to the field of tissue engineering, and more particularly to a tissue engineered vascular construct and a method for producing the same in vitro.

2. Background Art

Disorders of the vascular system result in significant morbidity and mortality and account for a large portion of health care expenditures. Coronary artery atherosclerotic disease and peripheral vascular disease are the most frequent causes of death in the United States. Surgical intervention in the form of vascular bypass grafting is commonly required. Additionally, a growing number of patients suffer from chronic renal failure necessitating hemodialysis, requiring procedures for vascular access such as arteriovenous loops implanted in the forearm. Furthermore, some patients undergoing microvascular plastic surgical procedures require small diameter bypass procedures to facilitate reconstruction.

Coronary artery bypass procedures are typically performed with the use of autologous vessels such as the greater saphenous vein, internal mammary artery, or radial artery. Lower extremity vascular bypass can be accomplished with the saphenous vein grafts, although expanded polytetrafluoroethylene (ePTFE) conduits have been used less successfully when adequate donor vein is not available. The current standard of care for long term vascular access for hemodialysis consists of using either native forearm vessels or ePTFE grafts to create high flow arteriovenous loops. The greater saphenous vein is often used for patients requiring vascular bypass for microvascular reconstructive procedures.

Harvest of native vessels for bypass procedures can produce donor site complications including infection, tissue loss, pain, and scarring. In addition, many patients, such as those who have undergone previous vascular bypass procedures, lack sufficient native vascular conduit suitable for grafting. Partly addressing these problems, surgeons sometimes use synthetic (prosthetic) materials such as Gor-Tex® (expanded polytetrafluoroethylene, ePTFE) or Dacron® (woven polyester). While synthetic grafts have seen wide application in large diameter reconstruction, the utility of synthetic conduits for small diameter (<3 mm) vessels has been limited by high thrombosis rates (see Jones et al., Cardiovasc Surg 5: 486-489, 1997; Graham et al., Surg Forum 30: 204-206, 1979).

Several vascular tissue engineering strategies have been tested in vivo. Most reported models employed grafts with internal diameters of 3 mm or larger, and most have used systemic anticoagulation to prevent graft occlusion with thrombosis. Kaushal et al. (Nat Med 7: 1035-1040, 2001) seeded 4 mm internal diameter acellularized porcine vessels with endothelial cells with a patency rate of 100% at 15 days when tested in sheep, where control non-seeded specimens had a 15 day patency rate of 25%. This study demonstrated that a recellularized graft could remain patent in vivo. L'Heureux et al. (FASEB J 12: 47-56, 1998) reported a tubed cell sheet, tested in a canine model, demonstrating the possibility of using an entirely cell-based approach. With the use of coumadin, aspirin, and systemic heparin, these grafts exhibited a 50% patency rate at one week. Niklason et al. (Science 284: 489-493, 1999) reported a study in which polyglycolic acid scaffolds were seeded with endothelial cells and smooth muscle cells and then implanted in Yucatan swine with daily aspirin treatment. The grafts measured 3 mm in internal diameter and had a four week patency rate of 100%. The grafts required two months of in vitro incubation in order to prepare them to withstand arterial pulsations. In a similar study, Shum-Tim et al. (Ann Thorac Surg 68: 2298-2304, 1999) reported a 100% 20 week patency rate in a 7 mm diameter ovine model in which vascular-derived cells were seeded onto a biodegradable scaffold. The grafts were implanted in the aortic position. Campbell et al. (Circ Res 85: 1173-1178, 1999) reported a study in which 3 mm diameter peritonealized mesothelial grafts had a patency of 67% at four months in a rat abdominal aortic interposition model. They showed that mesothelial cells can maintain graft patency in a small animal model. Huynh et al. (Nat Biotechnol 17: 1083-1086, 1999) reported the use of heparin-treated porcine small intestinal submucosa in a 4 mm internal diameter model implanted in rabbits, where the 12 week patency rate was 100%.

Although a substantial clinical need exists, there is currently no synthetic graft available to replace small (<3 mm diameter) vessels (see Kidd et al., J Surg Res 113: 234-242, 2003). A biologically-derived, tissue engineered vascular conduit may obviate many of the problems associated with the use of autologous and prosthetic grafts. The ideal tissue engineered vascular graft would be immunocompatible, resistant to thrombosis, readily available without a significant delay in development time prior to implantation, and would be mechanically compatible with the natural recipient vessels (see Edelman, Circ Res 85: 1115-1117, 1999; Nerem and Seliktar, Ann Rev Biomed Eng 3: 225-243, 2001; Mitchell and Niklason, Cardiovasc Pathol 12: 59-64, 2003; Skalak et al., Ann N.Y. Acad Sci 961: 255-257, 2002; Tranquillo, Ann N.Y. Acad Sci 961: 251-254, 2002; Nugent and Edelman, Circ Res 92: 1068-1078, 2003). Such a vascular graft would be of great value to patients undergoing free tissue transfer, coronary artery bypass, or lower extremity vascular reconstruction in cases of limited donor vessel availability.

### SUMMARY OF THE INVENTION

Accordingly, a small diameter vascular construct is provided which includes a vessel harvested from a donor and having an internal diameter of less than about 3 mm. The vessel is acellularized to remove cellular elements while retaining an extracellular matrix of the vessel. The vascular construct further includes endothelial cells provided in contact with the acellularized vessel, wherein the endothelial cells attach to the vessel to form the vascular construct.

In accordance with one aspect of the present invention, the vessel is acellularized chemically using at least one detergent solution. The acellularized vessel has a stiffness similar to native donor vessels and has low immunogenicity. Either the vessel or the endothelial cells can be harvested from cadaveric tissue, and the vessel and the endothelial cells can be harvested from different donors. The endothelial cells are provided in a suspension having a concentration of approximately 10⁷ cells/mL, and can be pipetted into a lumen of the acellularized vessel for seeding. In one embodiment, the internal diameter of the vessel is approximately 1 mm and the length of the vascular construct is about 5 to 10 mm.

Correspondingly, a method is provided for producing a vascular construct, where the method includes harvesting a vessel from a donor, the vessel having an internal diameter of less than about 3 mm. The method further includes acellularizing the vessel to remove cellular elements while retaining an extracellular matrix of the vessel, and seeding the acellularized vessel with endothelial cells to produce the vascular construct.

According to an aspect of the present invention, the method can further include banking the acellularized vessel for a period of time prior to seeding with the endothelial cells, and can also include immunohistochemically staining the endothelial cells to verify their origin. Still further, the method can include interpositionally grafting the vascular construct within a recipient vessel, where the grafting can be performed without the use of systemic anticoagulation.

In accordance with another aspect of the present invention, a method is provided for producing a vascular construct, where the method includes harvesting a vessel from a first donor, the vessel having an internal diameter of less than about 3 mm. The method further includes acellularizing the vessel to remove cellular elements while retaining an extracellular matrix of the vessel, and banking the acellularized vessel for a period of time. Still further, the method includes harvesting endothelial cells from a second donor, and seeding the acellularized vessel with endothelial cells to produce the vascular construct.

The above features and advantages and other features and advantages of the present invention are readily apparent from the following detailed description of the embodiments of the invention when taken in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a scanning electron micrograph of an acellularized vessel according to the present invention prior to recellularization;

FIGS. 2A-D are micrographs (20× objective lens) of an acellularized vessel stained with hematoxylin and eosin (FIG. 2A), immunofluorescently stained against elastin (FIG. 2B), immunoperoxidase labeled for von Willebrand factor (FIG. 2C), and stained for alpha smooth muscle actin (FIG. 2D), where the lumen is at the top of each image;

FIG. 3 is a micrograph (20× objective lens) of a recellularized vascular construct according to the present invention prior to implantation and stained for von Willebrand factor;

FIGS. 4A and 4B are photographs taken intraoperatively and four weeks postoperatively, respectively, of the gross morphology of a vascular construct graft (G) according to the present invention and the anastomosis with the native femoral artery (N);

FIGS. 5A-C are micrographs (20× objective lens) of vascular construct grafts explanted after four weeks in vivo, stained with hematoxylin and eosin (FIG. 5A), immunoperoxidase labeled for von Willebrand factor (FIG. 5B), and stained for alpha smooth muscle actin (FIG. 5C), where the lumen is at the top of each image; and

FIG. 6 is a graph of compression stress-strain curves of acellular vessel material (prior to endothelial cell seeding) and fresh vessels (prior to acellularization).

### DETAILED DESCRIPTION OF EMBODIMENT(S) OF THE PRESENT INVENTION

The present invention provides small diameter (<3 mm), tissue engineered vascular constructs and a method for producing the constructs in vitro. The method of the present invention includes acellularizing allograft donor vessels and subsequently recellularizing the vessels by incubation with a primary culture of endothelial cells, wherein this approach offers a number of advantages. First, as demonstrated below, the elastin fibers of the native donor vessels are maintained during acellularization, resulting in maintenance of an adequately robust extracellular matrix scaffold, resistant to arterial pressures. Second, the acellular vessel material can be prepared and banked in advance of endothelial seeding, offering the potential advantage of rapid production for clinical use. Third, acellular materials have been demonstrated to have low immunogenicity compared with cellular tissue, offering the theoretical advantage of negligible immune reactivity in vivo. Clinically, small diameter vascular constructs according to the present invention may be very useful in some cases of vascular reconstruction, such as small or distal coronary artery bypass, some microvascular reconstructive procedures, and in certain pediatric vascular reconstructions.

By way of example, the vascular construct and method for its production according to the present invention are described with reference to the use of vascular tissue harvested from and subsequently implanted in rats. However, it is fully contemplated that vascular tissue from any mammal, including human beings, could be similarly acellularized, recellularized, and grafted using the method described herein.

To produce vascular constructs according to the present invention, a vein or artery can be harvested from any location in the donor. Preferably, arteries are utilized, such as iliac arterial vessels from adult F344 rats (Harlan Sprague Dawley, Inc., Indianapolis, Ind.), where the harvested vessels have an internal diameter of less than 3 mm, and typically approximately 1 mm. Dissection under microscopic visualization is performed to remove the majority of the perivascular fat and connective tissue from the vessels. The vessels are pinned out to native in situ length on silicone-coated (Sylgard3, Dow Corning, Midland, Mich.) 100 mm polystyrene culture dishes using minutien pins. After harvesting, the vessels can be submersed in Dulbecco's Phosphate Buffered Saline (PBS) or another suitable balanced salt solution.

The acellularization method of the present invention is a chemical, rather than a mechanical, process that generally involves submersion of the donor vessel in a series of detergents and other reagents over a two week time period as shown in the table below. The acellularization process serves to remove the cellular elements of the vessel while leaving the connective framework intact, and thus produce immunologically inert residual tissue.

| Solution | Reagents | Time (Days) |
|---|---|---|
| 1 | Glycerol 80%/0.9% NaCl/0.05% | 3 |
| | NaN₃/25 mM EDTA | |
| 2 | Na deoxycholate 4.2%/0.05% NaN₃ | 3 |
| 1 | Glycerol 80%/0.9% NaCl/0.05% | 2 |
| | NaN₃/25 mM EDTA | |
| 3 | 1% SDS/0.05% NaN₃ | 2 |
| 4 | 3% Triton ® X-100/0.05% NaN₃ | 2 |
| 3 | 1% SDS/0.05% NaN₃ | 2 |
| 5 | 0.05% NaN₃ | 2 |

The acellularization process is typically carried out at room temperature (˜21° C.) within covered culture dishes. First, the vessels are submersed with Solution 1 for approximately 72 hours in order to disrupt the cell membrane. Second, the vessels are submersed with Solution 2 for approximately 72 hours to begin intracellular protein dissociation. Next, the vessels are again submersed with Solution 1, this time for approximately 48 hours to complete the removal of lipid-soluble cell structures. The vessels are then submersed with Solution 3 for approximately 48 hours for additional protein denaturing. Subsequently, the vessels are submersed with Solution 4 for approximately 48 hours in order to remove denatured proteins from the extracellular matrix, leaving the extracellular matrix intact. The vessels are next submersed with Solution 3 for approximately 48 hours to accomplish final protein denaturing and removal. Lastly, the vessels are submersed with Solution 5 where they can be stored until recellularization, wherein Solution 5 may be added as necessary to prevent evaporation.

Advantageously, the acellularization method of the present invention is simple, inexpensive, uses commonly available chemicals of low toxicity, and does not require mechanical agents of any kind. Furthermore, there should be no limitations on the length or diameter of vascular constructs that can be created, where larger diameter vessels can be acellularized by simply employing correspondingly longer immersion times for each solution. Of course, it is understood that all reagent measurements and submersion times described above are approximate, and can be varied slightly without affecting the resulting acellularization. The chemical acellularization method according to the present invention was utilized in the context of acellularizing muscle tissue in commonly assigned U.S. Pat. No. 6,207,451 and nerve tissue in U.S. Pat. No. 6,448,076, both of which are incorporated by reference herein.

According to the present invention, endothelial cells are harvested and expanded in vitro for use in recellularizing the acellularized vessels described above. Endothelial cells can be obtained from any donor tissue, typically vascular or blood tissue such as from adult F344 rat hearts. The harvested tissue is typically cut into ˜2 mm cubes and digested in 1% collagenase type I (Sigma-Aldrich, St. Louis, Mo.) at 37° C. for 30 minutes. This brief digestion of the cardiac tissue results in the preferential detachment of surface cells. Immunohistochemical staining for the endothelial cell surface marker von Willebrand factor can be performed to verify the endothelial origin of these cells, wherein purity is typically greater than 90%. The resulting cell suspension is collected for subculture and placed in a medium containing 45% Dulbecco's modification of Eagle's medium (DMEM, Invitrogen, Carlsbad, Calif.), 45% F12 medium (Invitrogen), 10% fetal bovine serum (Invitrogen), 0.5 μg/mL insulin (Sigma) and 1.0 μg/mL hydrocortisone (Sigma). Endothelial cells are passaged and subsequently maintained in a medium containing 20% FBS in DMEM. Cells used for seeding are typically passaged four times or more.

The acellularized vessels are cut into segments and soaked in DMEM for 24 hours at 37° C. prior to seeding with endothelial cells to allow diffusion of the sodium azide from the vessels. According to one aspect of the present invention, the length of the vessel segments can be approximately 5 to 10 mm. The endothelial cells are trypsinized and resuspended in 20% FBS in DMEM at a concentration of ˜10⁷ cells/mL, after which approximately 100 μL of the cell suspension (˜10⁶ cells) is gently pipetted into the lumen of each acellularized vessel. Of course, other concentrations and volumes of the endothelial cell suspension used for seeding are fully contemplated in accordance with the present invention. The resulting recellularized vascular constructs are then incubated overnight at 37° C. to allow for endothelial cell attachment, and can be maintained in an incubator until the vascular constructs are used for implantation.

To demonstrate the efficacy of the vascular constructs according to the present invention, the constructs were surgically implanted as interpositional grafts in the femoral arteries of isogenic adult F344 rats. Additionally, acellularized vessels that had not been recellularized were implanted in a separate group of animals as control grafts. Sections of the native vessel measuring 5 to 10 mm in length were removed, and replaced with vascular construct grafts or control grafts. Standard microsurgical techniques were employed, and anastomoses were performed with interrupted 10-0 nylon sutures. The graft lumens were irrigated with heparin solution (100 U/ml, Abbott Laboratories, Chicago, Ill.) during anastomosis, but no systemic anticoagulation was administered. The divided inguinal fat pat pad was reapproximated over the graft with absorbable sutures and the skin was closed with nylon sutures.

The vascular construct grafts and the control grafts were left in vivo for four weeks. This implantation period was chosen in order to evaluate intermediate to long-term patency rates as measured in previous small animal model studies (see Kidd et al., J Surg Res 113: 234-242, 2003). Postoperatively, patency of the grafts was monitored transcutaneously with a handheld pencil Doppler ultrasound (Parks Medical Electronics, Inc, Aloha, Oreg.) at the distal thigh, distal to the graft. Definitive assessment of patency was made by direct inspection of the grafts in vivo, including the detection of a Dopplerable arterial pulse and a positive “strip test” distal to each graft. In the “strip test,” blood was milked distally using forceps external to the graft, and another forceps occluded the vessel proximally. Patency was confirmed when the proximal forceps was released, filling the lumen of the graft with blood. In addition, the native vessels were transected distal to the vascular construct graft and the presence of arterial flow through the graft was noted.

Upon explantation at four weeks, direct examination confirmed the Doppler results in all cases. Despite the small internal diameter (1 mm) of the vascular construct grafts and the avoidance of systemic anticoagulation in the present invention, the vascular construct grafts had a four week patency rate of 89%, as compared with only 29% of the control grafts that remained patent. As such, the effect of pre-implantation recellularization with endothelial cells had a dramatic effect. These findings compare favorably with previous animal studies (see Huynh et al., Nat Biotechnol 17: 1083-1086, 1999; Kaushal et al., Nat Med 7: 1035-1040, 2001; Niklason et al., Science 284: 489-493, 1999; Campbell et al., Circ Res 85: 1173-1178, 1999) which tested grafts with larger internal diameters. The recellularization model according to the present invention is stringent from the standpoint of using a comparatively small internal diameter (1 mm), as opposed to 3 mm or more in previous studies, resulting in a cross-sectional area (πr²) of one-tenth that of previously reported models. Furthermore, the avoidance of systemic anticoagulation makes the model of the present invention more prothrombotic, and therefore more technically demanding than models that do use anticoagulation.

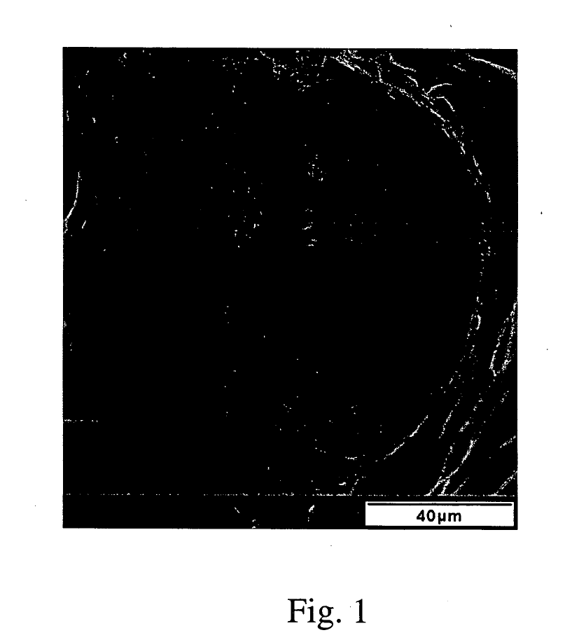

FIG. 1 is a scanning electron micrograph of an acellularized vessel prior to endothelial cell seeding. The acellular vessel material was loaded onto an ElectroScan E3 environmental scanning electron microscope (ElectroScan, Wilmington, Del.) and examined at a vapor pressure of 5 mm Hg. The scanning electron microscopy demonstrated the three dimensional architecture of the circumferentially arranged, obliquely oriented extracellular matrix fibers on the luminal surface of the acellularized vessel which remained structurally intact following the acellularization process.

Histologic analysis was performed on acellularized vessels prior to endothelial cell seeding, and on recellularized vascular constructs both pre- and post-implantation. The acellularized vessels and the vascular constructs were fixed in 4% formaldehyde (Fisher Scientific, Pittsburgh, Pa.), embedded in paraffin, transversely cut into 5 μm sections, and stained with hematoxylin and eosin (pressure fixation was not used). Immunohistochemistry was performed with immunoperoxidase-labeled antibodies against von Willebrand factor to localize endothelial cells, and against alpha smooth muscle actin to localize smooth muscle cells. Immunohistochemical staining for elastin was used to localize elastin fibers in the acellularized vessels. All primary and secondary antibodies were purchased from DakoCytomation, Inc., Carpinteria, Calif.

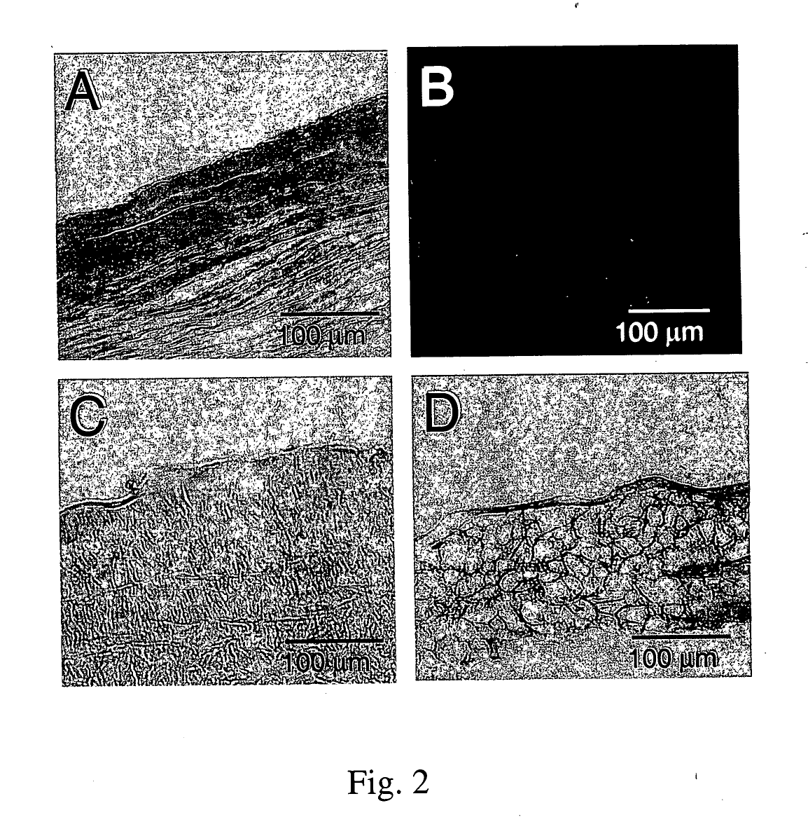

Referring to FIG. 2A, acellularized vessel sections stained with hematoxylin and eosin confirmed the absence of cells in the vessel wall following serial detergent treatment. Immunohistochemical staining for elastin indicated that multiple elastic laminae remained within the medial layer of the acellularized vessel after acellularization (FIG. 2B). FIG. 2B demonstrates that elastic fibers were present within the acellular vessel material, such that the constructs of the present invention were mechanically robust prior to implantation. This finding is important, since a vascular graft should contain elastic fibers in order to withstand cyclic deformation over the range of physiologic blood pressures while remaining compliant. Acellularized vessels did not stain positively for von Willebrand factor (FIG. 2C), and therefore did not contain endothelial cells. They also did not demonstrate positive staining for alpha smooth muscle actin, indicating a lack of smooth muscle cells (FIG. 2D).

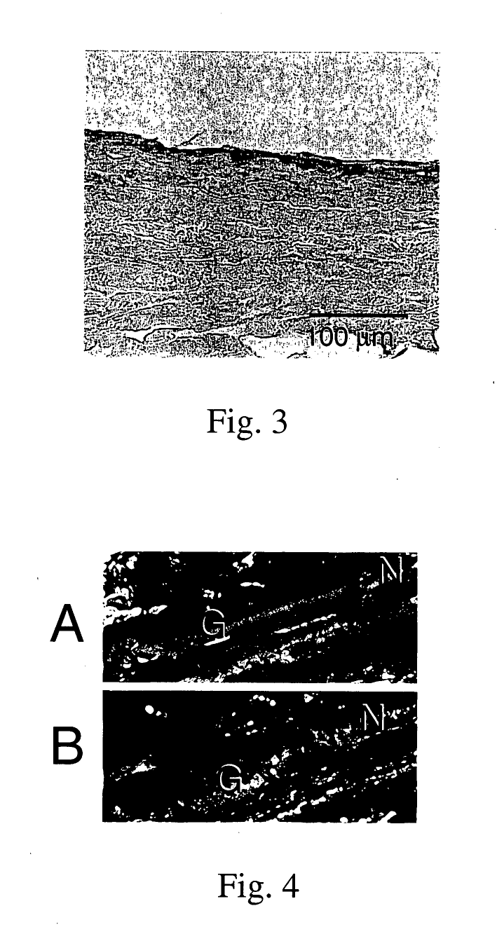

Contrary to the lack of endothelial cells present in the acellularized vessels prior to endothelial cell seeding (FIG. 2C), FIG. 3 depicts recellularized vascular constructs prior to implantation, wherein the constructs contained a layer of endothelial cells on their luminal surfaces as demonstrated by red-brown von Willebrand factor immunostaining.

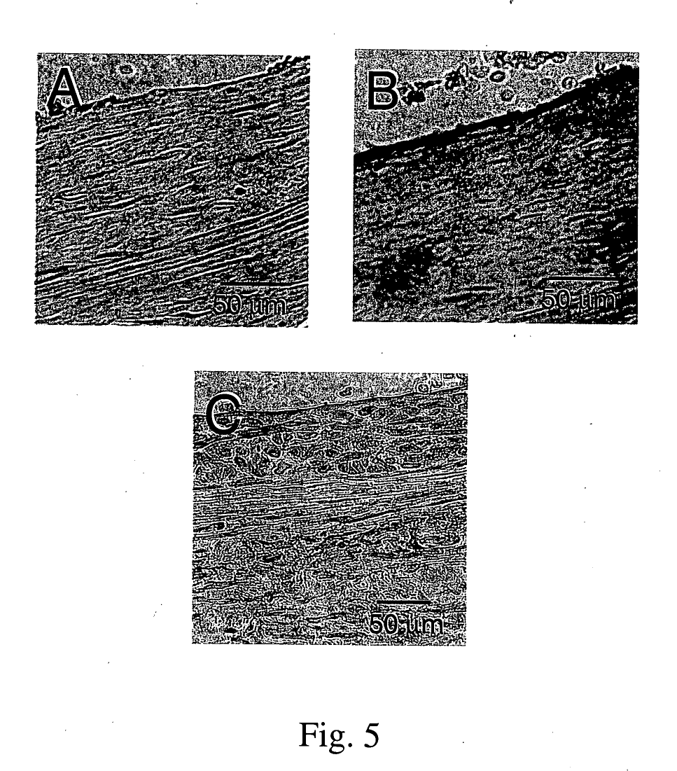

FIGS. 4A and 4B illustrate the gross morphology of a vascular construct graft (G) according to the present invention and the anastomosis with the native femoral artery (N) both intraoperatively and four weeks postoperatively, respectively. Turning to FIGS. 5A-5C, following four weeks of implantation, sections of vascular construct grafts according to the present invention stained with hematoxylin and eosin demonstrated that abundant cellular infiltration was present throughout the entire substance of the grafts (FIG. 5A). Vascular construct graft sections stained for von Willebrand factor demonstrated an intact layer of endothelial cells on the luminal surface (red-brown staining; FIG. 5B). While absent in the acellularized vessels prior to implantation (FIG. 2D), smooth muscle cells appeared to have migrated into the vascular construct grafts during their period of in vivo implantation, as smooth muscle actin staining demonstrated the presence of smooth muscle cells within the walls of the vascular construct grafts post-implantation in the grafts that remained patent (red-brown staining; FIG. 5C).

The origin of the endothelial cells in the explanted grafts is unknown. They may be the original endothelial cells seeded into the vascular constructs prior to implantation, or, more likely, a mix of the original endothelial cells with endogenous (host-derived) endothelial cells. Likewise, the origin of the smooth muscle cells present in the vascular construct grafts at the time of explantation is not known. While the most straighforward explanation would be migration of adjacent native smooth muscle cells into the vascular construct graft, some have proposed that circulating precursors may be able to differentiate into smooth muscle cells and seed the grafts (see Shimizu et al., Nat Med 7: 738-741, 2001).

The uniaxial stress-strain response of the acellularized vessels (prior to seeding with endothelial cells) was examined in compression and the mechanical properties were compared with those of fresh vessels (prior to acellularization). Rectangular specimens measuring approximately 2.0 mm by 2.0 mm by 0.15 mm (thickness) were cut from three acellularized vessels and also from three fresh vessels. The specimens were stored in saline solution at 22° C. for up to three hours prior to testing. Uniaxial compression tests were conducted such that the compression vector was perpendicular to the vessel wall. An MTS 810 servohydraulic test system (MTS Systems Corp., Eden Prarie, Minn.) was used to provide a constant true strain rate test at 0.01/sec to a maximum compressive strain of 0.3. The load vs. displacement data were collected with a National Instruments DAC (Austin, Tex.) and a PC for data storage and manipulation. These data were converted to nominal stress (load divided by initial cross section) versus nominal strain (change in length divided by original length). The maximum value of the tangent stiffness was determined as the maximum slope of the stress vs. strain response during compression loading.

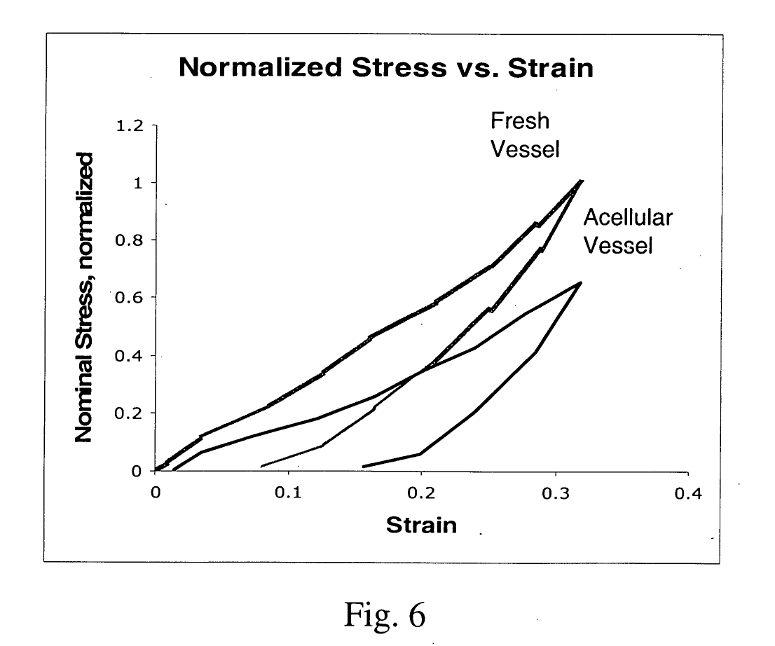

Stress vs. strain response curves for acellularized vessels and fresh tissue are shown in FIG. 6. Both curves were normalized by the maximum value of nominal stress for all tests. The uniaxial compression response of both tissues was similar; the tissues displayed characteristic non-linearity during loading and hysteresis upon unloading of soft tissue. The tangent stiffnesses for these two curves were calculated at the maximum recorded nominal strain of 0.275. The tangent stiffnesses of the three fresh vessels were averaged and compared to the average value of the three acellularized vessels. The maximum value of the averaged acellularized vessel tangent stiffness was 2.147±0.629 kPa, which was equivalent to 81% of that of the fresh vessels, whose average maximum tangent stiffness was 2.654±1.135 kPa.

Mechanical similarity between graft and recipient vascular conduits has been shown to be important in preventing intimal hyperplasia, occlusion, and aneurysm formation (see Berglund et al., Biomaterials 24: 1241-1254, 2003; Nerem, Biorheology 40: 281-287, 2003; Nerem, J Biomech Eng 115: 510-514, 1993; Nerem, Biorheology 21: 565-569, 1984; Ziegler and Nerem, J Cell Biochem 56: 204-209, 1994; Perktold et al., Ann Biomed Eng 30: 447-460, 2002). Compliance mismatch (a significant difference in elastic modulus, or tangent stiffness, between vascular conduits) is thought to be a major factor in the increased rate of occlusion seen in prosthetic vascular grafts. The presence of elastic fibers in an engineered vascular graft should improve the graft's ability to withstand cyclic deformation over a range of physiologic blood pressures, while remaining compliant. Elastic fibers are present within the acellular vessel material (FIG. 3B), the vascular construct grafts of the present invention remained mechanically robust (i.e., withstood cyclic deformation under arterial pressures without failure) during their four weeks in vivo. Compression loading demonstrated that the tangent stiffnesses of the acellular vessel material were similar to those of fresh vessels. The compression test described herein provides the same deformation state in the tissue that it experiences during pressurization in vivo. Ductile materials such as soft tissue fail when a critical value of an appropriate scalar measure of the strain tensor is exceeded, and the compression test performed herein establishes that the acellularized vessels and resulting vascular constructs of the present invention can withstand the strain state and strain magnitudes seen in vivo.

In addition to in vivo patency rates and mechanical considerations, the time required for production of vascular constructs should be addressed in evaluating strategies for vascular tissue engineering. A short production time is distinctly advantageous for clinical application of these products, as most patients would be unable to wait several weeks or months for a coronary bypass, for example, while their vessels were being prepared in vitro. Many approaches, including cell self-assembly, cell-seeded collagen gels, biodegradable synthetic polymer scaffolds, and acellular techniques require weeks to months of incubation in order to generate de novo extracellular matrix capable of withstanding arterial pulsations in vivo (see Nerem and Seliktar, Ann Rev Biomed Eng 3: 225-243, 2001; Mitchell and Niklason, Cardiovasc Pathol 12: 59-64, 2003; Niklason et al., Science 284: 489-493, 1999; Campbell et al., Circ Res 85: 1173-1178, 1999; Berglund et al., Biomaterials 24: 1241-1254, 2003; Ziegler and Nerem, J Cell Biochem 56: 204-209, 1994; Gao et al., J Biomed Mater Res 42: 417-424, 1998; Niklason and Langer, Transpl Immunol 5: 303-306, 1997; Niklason et al., J Vasc Surg 33: 628-638, 2001; Nerem, Yonsei Med J 41: 735-739, 2000; Black et al., FASEB J 12: 1331-1340, 1998; L'Heureux et al., FASEB J 15: 515-524, 2001; L'Heureux et al., FASEB J 12: 47-56, 1998; L'Heureux et al., J Vasc Surg 17: 499-509, 1993).

The use of an acellularized vascular matrix as in the present invention confers the advantage of mechanical robustness from the outset, rather than waiting weeks or months for pre-conditioning measures to stimulate the production of extracellular matrix by cells within the construct. Acellularized vessel scaffolds also potentially allow for relatively short production times, since the acellularized vessels can be manufactured in advance of the anticipated need. The acellularized vessels of the present invention can be generated from organ or tissue donors, and then banked so as to be ready for endothelial seeding whenever the need arises. Advantageously, the low immunogenicity of the acellularized vessels of the present invention means that the banked vessels need not be from the recipient, nor do the banked vessels need to have the same donor as the endothelial cells used for seeding. This also allows for the use of cadaveric tissues in preparing either the acellularized vessel or endothelial cells.

In summary, the present invention demonstrates that the vascular constructs of the present invention remain patent when implanted in vivo, exhibit compatible mechanical properties with recipient tissue, and structurally and histologically resemble native arteries post-implantation. These vascular constructs have implications for future studies in vascular tissue engineering and vascular biology, and may prove suitable for use as small vessel vascular graft material, including coronary and lower extremity vascular bypass, long term hemodialysis access, or as additional conduit for plastic surgical free tissue transfer procedures.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

1. A small diameter vascular construct, comprising:
a vessel harvested from a donor and having an internal diameter of less than about 3 mm, the vessel acellularized to remove cellular elements while retaining an extracellular matrix of the vessel; and
endothelial cells provided in contact with the acellularized vessel, wherein the endothelial cells attach to the vessel to form the vascular construct.

2. The vascular construct according to claim 1, wherein the vessel is acellularized chemically using at least one detergent solution.

3. The vascular construct according to claim 1, wherein the vessel is an artery.

4. The vascular construct according to claim 1, wherein the endothelial cells are harvested from one of vascular and blood tissue.

5. The vascular construct according to claim 1, wherein the vessel and the endothelial cells are harvested from different donors.

6. The vascular construct according to claim 1, wherein at least one of the vessel and the endothelial cells are harvested from cadaveric tissue.

7. The vascular construct according to claim 1, wherein the acellularized vessel has low immunogenicity.

8. The vascular construct according to claim 1, wherein the acellularized vessel can be banked for a period of time prior to providing the endothelial cells.

9. The vascular construct according to claim 1, wherein the endothelial cells are provided in a suspension having a concentration of approximately 10⁷ cells/mL.

10. The vascular construct according to claim 1, the endothelial cells are pipetted into a lumen of the acellularized vessel.

11. The vascular construct according to claim 1, wherein the internal diameter of the vessel is approximately 1 mm.

12. The vascular construct according to claim 1, wherein the length of the vascular construct is about 5 to 10 mm.

13. The vascular construct according to claim 1, wherein the acellularized vessel has a stiffness similar to native donor vessels.

14. A small diameter vascular construct, comprising:
a vessel harvested from a donor and having an internal diameter of approximately 1 mm, the vessel chemically acellularized to remove cellular elements while retaining an extracellular matrix of the vessel; and
endothelial cells provided in contact with a lumen of the acellularized vessel, wherein the endothelial cells attach to the vessel to form the vascular construct.

15. A method of producing a vascular construct, comprising:
harvesting a vessel from a donor, the vessel having an internal diameter of less than about 3 mm;
acellularizing the vessel to remove cellular elements while retaining an extracellular matrix of the vessel; and
seeding the acellularized vessel with endothelial cells to produce the vascular construct.

16. The method according to claim 15, wherein acellularizing the vessel includes chemically acellularizing the vessel using at least one detergent solution.

17. The method according to claim 15, wherein harvesting the vessel includes harvesting an artery.

18. The method according to claim 15, further comprising harvesting the endothelial cells from one of vascular and blood tissue.

19. The method according to claim 15, further comprising harvesting the vessel and the endothelial cells from different donors.

20. The method according to claim 15, further comprising banking the acellularized vessel for a period of time prior to seeding with the endothelial cells.

21. The method according to claim 15, further comprising immunohistochemically staining the endothelial cells to verify their origin.

22. The method according to claim 15, wherein seeding the acellularized vessel includes providing the endothelial cells in a suspension having a concentration of approximately 10⁷ cells/mL.

23. The method according to claim 15, wherein seeding the acellularized vessel includes pipetting the endothelial cells into a lumen of the acellularized vessel.

24. The method according to claim 15, wherein the internal diameter of the vessel is approximately 1 mm.

25. The method according to claim 15, wherein the length of the vascular construct is about 5 to 10 mm.

26. The method according to claim 15, further comprising interpositionally grafting the vascular construct within a recipient vessel.

27. The method according to claim 26, wherein grafting is performed without the use of systemic anticoagulation.

28. A method of producing a vascular construct, comprising:
harvesting a vessel from a first donor, the vessel having an internal diameter of less than about 3 mm;
acellularizing the vessel to remove cellular elements while retaining an extracellular matrix of the vessel;
banking the acellularized vessel for a period of time;
harvesting endothelial cells from a second donor; and
seeding the acellularized vessel with endothelial cells to produce the vascular construct.
